(19) United States
(12) Patent Application Publication
(10) Pub. No.: US 2018/0035868 A1
(43) Pub. Date: Feb. 8, 2018

(54) IMAGING DEVICE, ENDOSCOPE, AND ENDOSCOPE SYSTEM

(71) Applicant: OLYMPUS CORPORATION, Tokyo (JP)

(72) Inventor: Masashi Saito, Tokyo (JP)

(73) Assignee: OLYMPUS CORPORATION, Tokyo (JP)

(21) Appl. No.: 15/786,799

(22) Filed: Oct. 18, 2017

Related U.S. Application Data
(63) Continuation of application No. PCT/JP2015/062393, filed on Apr. 23, 2015.

(51) Int. Cl. A61B 1/00; A61B 1/06

(52) U.S. Cl. CPC A61B 1/00009; A61B 1/00045; A61B 1/0661; A61B 1/00018; A61B 1/05

(57) ABSTRACT

In an imaging device, a pixel signal processing circuit outputs an imaging signal based on a pixel signal. A level shift circuit shifts a first level of the imaging signal in a direction in which the first level is away from a second level of a reference signal or shifts the second level in a direction in which the second level is away from the first level. A signal output terminal outputs the reference signal and the imaging signal, the first level of which is shifted, to an imaging signal processing circuit or outputs the reference signal the second level of which is shifted, and the imaging signal. The imaging signal processing circuit calculates a difference between the reference signal and the imaging signal output from the signal output terminal.

### FIELD OF THE INVENTION

The present invention relates to an imaging device, an endoscope, and an endoscope system.

Priority is claimed on PCT International Patent Application No. PCT/JP2015/062393, filed Apr. 23, 2015, the content of which is incorporated herein by reference.

### DESCRIPTION OF RELATED ART

In the related art, imaging devices such as a complementary metal-oxide semiconductor (CMOS) image sensor hold imaging signals transferred for each row of a plurality of pixels in sample-and-hold circuits. In addition, imaging devices sequentially output held imaging signals to horizontal output signal lines for each pixel . Analog front end circuits provided outside imaging devices can calculate differences between reference signals (power supply voltages) and imaging signals to generate the imaging signals in which fixed pattern noise of the imaging devices is reduced.

Japanese Unexamined Patent Application, First Publication No. 2006-121652 discloses a technology of reducing a noise component due to joule heat. generated in a pn junction in an infrared sensor. In such a technology, a difference between a. voltage of a signal including a valid signal. and a voltage of a reference signal including a noise component is calculated on the basis of the same principle as correlated double sampling (CDS) in an imaging device. Such a technology reduces the noise component using the^{,} same method as a technology of reducing a fixed pattern noise of the imaging device.

### SUMMARY OF THE INVENTION

According to a first aspect of the present invention, an imaging device includes a plurality of pixels, a pixel signal processing circuit, a reference signal generation circuit, a level shift circuit, and a signal output terminal. The plurality of pixels output pixel signals. The pixel signal processing circuit processes each of the pixel signals and outputs an imaging signal based on the pixel signal. The reference signal generation circuit generates a reference signal. The level shift circuit shifts a first level of the imaging signal in a direction in which the first level is away from a second level of the reference signal. Alternatively, the level shift circuit shifts the second level in a direction in which the second level is away from the first level. The signal output terminal outputs the reference signal generated by the reference signal generation circuit and the imaging signal, the first level of which is shifted by the level shift circuit, to an imaging signal processing circuit. Alternatively, the signal output terminal outputs the reference signal, the second level of which is shifted by the level shift circuit, and the imaging signal output from the pixel signal processing circuit to the imaging signal processing circuit,. The imaging signal processing circuit calculates a difference between the reference signal and the imaging signal output from the signal output terminal.

According to a second aspect of the present invention, in the first aspect, a relationship between levels of the reference signal and the imaging signal output from the signal output terminal when light is not incident on the plurality of pixels may he the same as a relationship between levels of the reference signal and the imaging signal output from the signal output terminal When light is incident on the plurality of pixels.

According to a third aspect of the present invention, in the first aspect, a difference of levels of the reference signal and the imaging signal output from the signal output terminal when light is not incident on the plurality of pixels may be within 20% of the maximum value of a difference between levels of the reference signal and the imaging signal which can he output from the signal output terminal.

According to a fourth aspect of the present invention, in the first aspect, the imaging device may further include a reference voltage generation circuit configured to generate a reference voltage used to operate the pixel signal processing circuit, The reference signal generation circuit may generate the reference signal from the reference voltage.

According to a fifth aspect of the present invention, an endoscope includes an insertion unit configured to he inserted into a subject and the imaging device disposed on. a tip of the insertion unit.

According to a sixth aspect of the present invention, an endoscope system. includes an endoscope the imaging signal processing circuit and an image signal generation circuit, The image signal generation circuit processes a difference signal based on the difference calculated by the imaging signal processing circuit and generate an image signal based on the difference signal.

### BRIEF DESCRIPTION OF THE DRAWINGS

FIG. 1 is a schematic diagram showing a configuration of an endoscope system according to an embodiment of the present invention.

FIG. 2 is a block diagram showing the configuration of the endoscope system according to the embodiment of the present invention.

FIG. 3 is a block diagram showing a configuration of a first chip in the endoscope system according to the embodiment of the present invention.

FIG. 4 is a circuit diagram of the first chip in the endoscope system according to the embodiment of the present invention.

FIG. 5 is a circuit diagram of a reference current source in the endoscope syst CM according to the embodiment of the present invention.

FIG. 6 is a circuit diagram of a reference current source in the endoscope system according to the embodiment of the present invention.

FIG. 7 is a timing chart for describing an operation of an imaging unit in the endoscope system according to the embodiment of the present invention.

FIG. 8 is a block diagram showing a configuration of a first chip in an endoscope system of a modified example according to the embodiment of the present invention.

FIG. 9 is a circuit diagram of the first chip in the endoscope system of the modified. example according to the embodiment. of the present invention,

### DETAILED DESCRIPTION OF THE INVENTION

An embodiment of the present invention will be described with reference to the drawings.

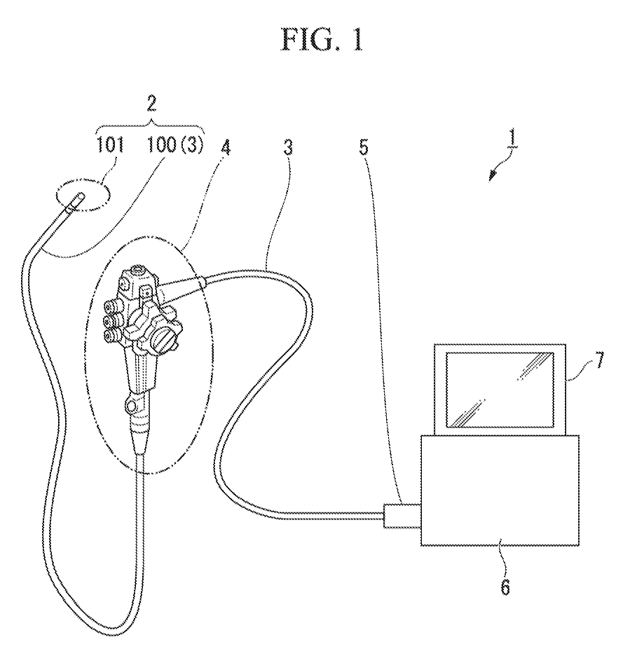

FIG. 1 shows a configuration of an endoscope system I according to the^{,} embodiment of the present inventionAs shown in FIG. 1, the endoscope system 1 includes an endoscope 2, a transmission cable 3, an operation unit 4, a connector unit 5, a processor 6, and a display device 7.

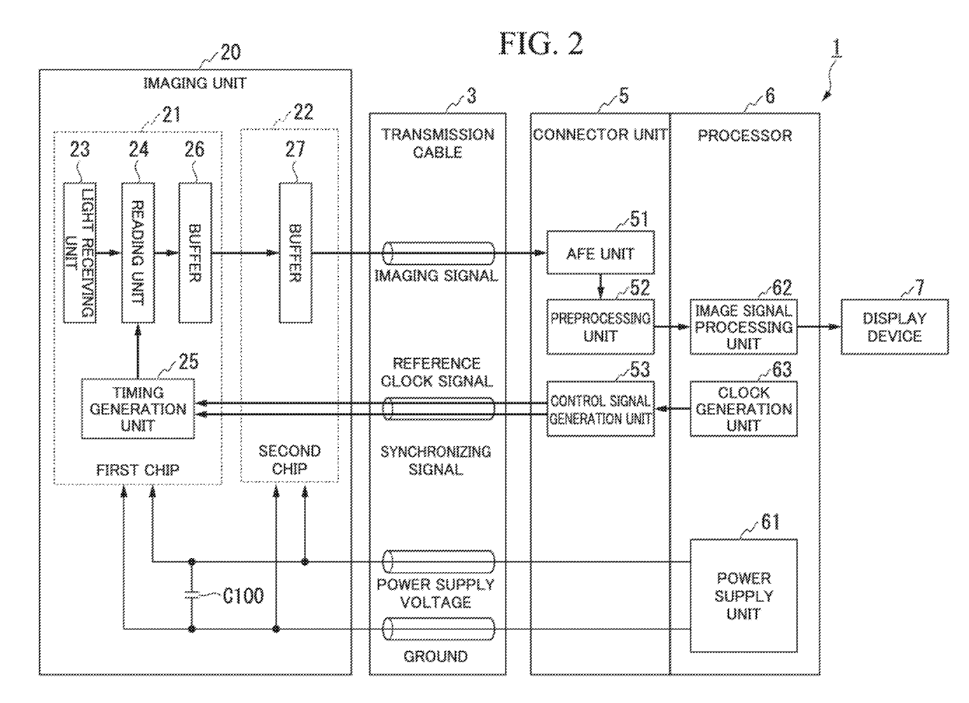

The endoscope 2 includes an insertion unit 100 inserted into a subject. The insertion unit 100 is a part of the transmission cable 3. The insertion unit 100 is inserted into a subject. The endoscope 2 generates an imaging signal (image data) by imaging an inside of the subject. The endoscope 2 outputs the generated imaging signal to the processor 6. An imaging unit 20 (an imaging device) shown in FIG. 2 is disposed on a tip 101 of the insertion unit 100. In the insertion unt 100, the operation unit 4 is connected to an end portion which is opposite to the tip 101. The operation unit 4 receives various operations performed on the endoscope 2.

The transmission cable 3 connects the imaging unit 20 and the connector unit 5 of the endoscope 2. An imaging signal generated by the imaging unit 20 is output to the connector unit 5 via the transmission cable 3.

The connector unit 5 is connected to the endoscope 2 and the processor 6. The connector unit. 5 pertbrms a predetermined signal processing on the imaging signal output from the endoscope 2, hi addition, the connector unit. 5 performs

The connector unit 5 outputs the digital image signal to the processor 6.

The processor 6 pertbrms a predetermined image processing on an image signal output from the connector unit 5. In addition, the processor 6 comprehensively controls the entire endoscope system 1.

The display device 7 displays an image corresponding to the image signal processed by the processor 6. In addition, the display device 7 displays various kinds of information associated with the endoscope system 1.

The endoscope system I includes a light source device configured to generate illumination light radiated on a subject. In FIG. 1, the light source device will be omitted.

FIG. 2 shows a configuration of an inside of the endoscope system 1. As shown in FIG. 2, the endoscope system I includes the imaging unit 20, the transmission cable 3, the connector unit 5, and the processor 6.

The imaging unit 20 includes a first chip 21 (an imaging element) and a second chip 22. The first chip 21 includes a light receiving unit 23, a reading unit 24, a timing generation unit 25, and a buffer 26. The imaging unit 20 functions as an imaging device.

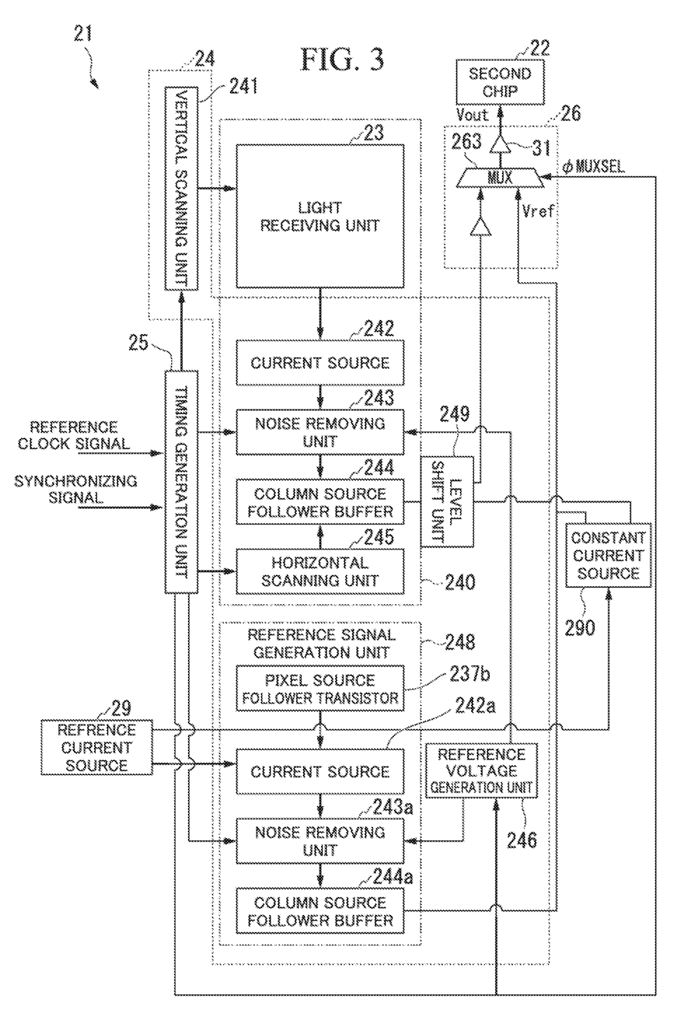

The light receiving unit 23 includes a plurality of pixels and generates an generated by the light receiving unit 23. In addition, the reading unit 24 generates a reference signal. The timing generation unit. 25 generates a timing signal on the basis of a reference clock signal and a synchronizing signal output from the connector unit 5. The timing signal generated by the timing generation unit 25 is output to the reading unit 24. The reading unit 24 reads the imaging signal in accordance with the timing signal. The buffer 26 temporarily holds the imaging signal and a reference signal read from the light receiving unit 23. A more detailed configuration of the first chip 21. will be described below with reference to FIG. 3.

The second chip 22 includes a buffer 27. The buffer 27 outputs the imaging signal output from the first chip 21 to the connector unit 5 via the transmission cable 3. A combination of circuits mounted in the first chip 21 and the second chip 22 can be changed appropriately in accordance with settings.

A power supply voltage and a ground voltage generated by the processor 6 are transmitted to the imaging unit 20 through the transmission cable 3. In the imaging unit 20, a capacitor C100 for power supply stabilization is disposed between a signal line configured to transmit the power supply voltage and a signal line configured to transmit the ground voltage.

The connector unit 5 includes an analog front end unit 51 (hereinafter referred to as an “AFE unit 51”), a preprocessing unit 52, and a control signal generation unit 53. The connector unit 5 electrically connects the endoscope 2 (the imaging unit. 20) and the processor 6. The connector unit 5 and the imaging unit 20 are connected through the transmission cable 3. The connector unit 5 and the processor 6 are connected through a coil cable.

The AFE unit 51 (an imaging signal processing circuit) calculates a ditTerence between a reference signal and an imaging signal. In addition, the AFE unit 51 performs AID conversion on the imaging signal based on the difference. The ME unit 51. outputs the imaging signal converted into a digital signal through the AID conversion to the preprocessing unit 52,

The preprocessing unit 52 perforins a predetermined signal processing such as vertical line removal and noise removal on the digital imaging signal output from the

AFE unit 51. The preprocessing unit 52 outputs the imaging signal which has been subjected to the signal processing to the processor 6.

A reference clock signal serving as a reference of an operation of each unit of the endoscope 2 is supplied from the processor 6 to the control signal generation unit 53. For example, a frequency of the reference clock signal is 27 MHz. The control signal generation unit 53 generates a synchronizing signal indicating a start position of each frame on the basis of the reference clock signal. The control signal generation unit 53 outputs the reference clock signal and the synchronizing signal to the timing generation unit 25 of the imaging unit 20 via the transmission. cable 3. The synchronizing signal generated by the control signal generation unit 53 includesa horizontal synchronizing signal and a vertical synchronizing signal.

The processor 6 is a control device which totally controls the entire endoscope system 1. The processor 6 includes a power supply unit 61. an image signal processing unit 62, and a clock generation unit 63.

The power supply unit 61 generates a power supply voltage. The power supply unit 61. outputs the power supply voltage and a ground voltage to the imaging unit 20 via the connector unit 5 and the transmission cable 3.

The image signal processing unit 62 (an image signal generation circuit) performs a predetermined image processing on a digital imaging signal processed by the preprocessing unit 52. The predetermined image processing may include a. synchronization process, a white balance (WB) adjustment process, a gain adjustment process, a gamma correction process, a digital-to-analog (D/A) conversion process, a format conversion process, and the like. The image signal processing unit 62 converts an imaging signal into an image signal using such an image processing. In other words, the image signal processing unit 62 processes the imaging signal (a difference signal) based on a difference calculated by the AFT unit 51 and generates the image signal hared on the imaging signal. The image signal processing unit 62 outputs the generated image signal to the display device 7.

The clock generation unit 63 generates a reference clock serving as a reference of an operation of each unit of the endoscope system 1. The clock generation unit 63 outputs the generated reference clock signal to the control signal generation unit 53 .

The display device 7 displays an image captured by the imaging unit 20 on the basis of an. image signal output from the image signal processing unit 62. The display device 7 includes a display panel such as a liquid crystal or organic electro luminescence (EL).

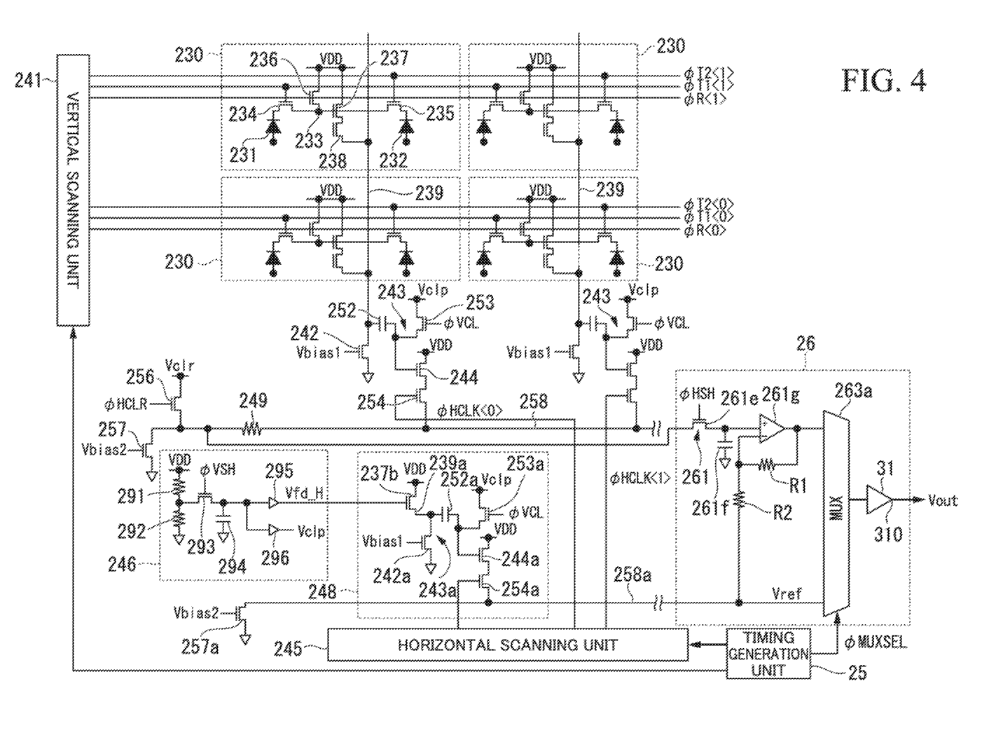

A detailed configuration of the first chip 21 will he described. FIG. 3 shows the^{,} configuration of the first chip 21. FIG. 4 shows a circuit configuration of the first chip 21 .As shown in FIGS. 3 and 4, the first chip 21 includes the light receiving unit 23, the reading unit 24, the timing generation unit 25, the buffer 2h, a reference current source 29. and a constant current source 290.

A reference clock signal and a synchronizing signal generated by the control signal generation unit 53 are input to the timing generation unit 25. The timing generation unit 25 generates various types of control signals on the basis of the reference clock signal and the synchronizing signal. The timing generation unit 25 outputs the generated control signal to a vertical scanning unit 241 of the reading unit 24, a noise removing unit 2.43. a horizontal scanning unit 245, the noise removing unit 243a of a reference signal generation unit 248, and a multiplexer 263a of the buffer 26.

The light receiving unit 23 includes a plurality of pixels 230 configured to output an imaging signal. FIG. 4 shows four representative pixels 230. The reading unit 24 reads an imaging signal output from each of the plurality of pixels 230 of the light receiving unit 23 and a reference signal output from the reference signal generation unit 248. A period in which the imaging signal is read is different from a period in which the :eference signal is read. The reading unit 24 transfers the read imaging signal and reference signal to the buffer .26.

A detailed configuration of the reading unit 24 will he described. The reading unit 24 includes the vertical scanning unit 241 (a row selection circuit), a current source 242, the noise removing unit 243 (a pixel signal processing circuit), a column source follower buffer 244, a horizontal scanning unit 245, a reference voltage generation unit 246 (a reference voltage generation circuit), the reference signal generation unit 248 (a reference signal generation circuit), and a level shift unit 249 (a level shift circuit).

The vertical scanning unit 241 outputs a control signal (lift <M.>(M =0. 1. 2, -m-1, and m), a control signal oT2^{,-}A1 , and a control signal (I)R<M>on the basis of a control signal input from the timing generation unit 25. The control signal gal<M>, the control signal 4^{,}T2<M>and the control signal 4R<M>are output to the pixels 230 of a row <M>selected from the plurality of pixels 230 of the light receiving unit 23. The plurality of pixels 230 output pixel signals and noise signals to vertical transfer lines 239, Each of the pixel signals includes a component based on light. which is incident on the pixels 230. Each of the noise signals includes a signal variation in accordance with the plurality of pixels 230 and noise when each of the pixels 230 is reset. The vertical transfer lines 239 are disposed in a column direction of the plurality of pixels 230 of the light receiving unit 23. The vertical transfer lines 239 are disposed to correspond to a plurality of columns of the plurality of pixels 230 of the light receiving unit 23. The pixel signal and the noise signal aretransferred to the noise removing unit 243 through each of the vertical transfer lines 239.

The noise emoving unit 243 generates an imaging signal corresponding to a. difference between the pixel signal and the noise signal. In other words, the noise removing unit 243 removes a signal variation in accordance with the plurality of pixels 230 and a noise when the pixels 230 are reset from a pixel signal. Thus, the noise removing unit 243 outputs an imaging signal based on a component in accordance with. light which is incident on the plurality of pixels 230. Details of the noise .removing unit 243 will he described below.

The horizontal scanning unit 245 outputs a control signal .01.10.,K<N (N =0, 1, 2 . - - -, rt--.1, and n) on the basis of a control signal supplied from the timing generation unit 25. The control signal iᵢSFICLIK<N>is output to a reading circuit corresponding to a column <N>selected from the plurality of pixels 230 of the light receiving unit 23. The imaging signal processed by the noise removing unit 243 is transferred to a horizontal transfr line 258 via the reading circuit. The horizontal transfer line 258 is disposed in a row direction of the plurality of pixels 230 of the light receiving unit 23. The imaging signal is transferred to the buffer 26 through the horizontal transfer line 258.

A detailed configuration of the light receiving unit 23 will be described. The light receiving unit 23 includes the plurality of pixels 230 disposed in a two-dimensional matrix form. The plurality of pixels 230 include a photoelectric conversion element 231 is photodiode), a photoelectric conversion element 232, a charge conversion unit2:7,3, a transfer transistor 234, a transfer transistor 2 35, a pixel reset transistor 236, a pixel source fbllovver transistor 237, and a selection transistor 238. The light receiving unit 23. the current source 242, the noise removing unit 243, the columnsourcefollower butler 244, and the horizontal scanning unit 245 function as an imaging signal generation unit 240. The imaging signal generation unit 240 generates pixel signals by converting electric charges accumulated in a plurality of photoelectric conversion elements 231 and a plurality of photoelectric conversion elements 232 into voltages. Each of the photoelectric conversion elements 231 and the photoelectric conversion elements 232 includes a first terminal and a second terminal. The first terminal of the photoelectric conversion element 231 is connected to a ground. The second terminal of the photoelectric conversion element 231. is connected to a first terminal of the transfer transistor 234. The first terminal of the photoelectric conversion element 232 is connected to the ground. The second terminal of the photoelectric conversion element 232. is connected to a first terminal of the transfer transistor 235. The photoelectric conversion element 231 and the photoelectric conversion element 232 receive light from the outside and accumulate electric charges according to an amount of received light.

The charge conversion unit 233 is constituted of a floating diffusion capacitance (floating diffusion). The charge conversion unit 233 converts electric charges accumulated in the photoelectric conversion element 231 and the photoelectric conversion element 232 into voltages.

The transfer transistor 234 includes a first terminal, a second terminal, and a. gate. The first terninal and the second terminal of the transfer transistor 234 are a source or a drain. The first terminal of the transfer transistor 234 is connected to the second terminal of the photoelectric conversion element 231. The second terminal of the transfer transistor 234 is connected to the charge: conversion unit 233. A control.

signal 4Tl is supplied from the ertical scanni tit 241 to the gate of the transfer transistor 234. The transfer transistor 234 is switched on *hen receiving the control signal T l from the vertical scanning unit 241. Thus, the transfir transistor 234 transfers an ectric charge from the photoelectric conversion element 231 to the charge conversion unit 233.

The transfer transistor 235 includes a first terminal, a second terminal, and a gate. The first terminal and the second terminal of the transfer transistor 235 are a source or a drain. The first terminal of the transfer transistor 235 is connected to the second terminal of the photoelectric conversion element 232 . The second terminal of the transfer transistor 235 is connected to the charge conversion unit 233. A control signal 0′2 is supplied from the vertical scanning unit 241 to the gate of the transfer transistor 235. The transfer transistor 235 is switched on when receiving the control signal 02 from the vertical scanning unit 241. Thus, the transfer transistor 235 transfers an electric charge from the photoelectric conversion element 232 to the charge conversion unit 233. At this time, a pixel signal is generated.

The pixel reset transistor 236 includes a first terminal, a second terminal, and a gate. The first tel urinal and the second terminal of the pixel reset transistor 236 are a source or a drain. A power supply voltage VDD is input to the first terminal of the pixel reset transistor 236. The second terminal of the pixel reset transistor 236 is connected to the charge conversion unit 233. A control signal OR is suppled from the vertical scanning unit 241 to the gate of the pixel reset transistor 236. The pixel reset transistor 236 is switched on when receiving the control signal 4R from the vertical scanning unit 241. Thus, the pixel reset transistor 236 resets a potential of the charge conversion unit 233 to a. predetermined potential. At this time, the pixels 230 are reset, and a noise signal is generated. terminal, and a gate. The first terminal and the second terminal of the pixel source follower transistor 237 are a source or a drain. The power supply voltage Vl)l) is input to the first terminal of the pixel source f011ower transistor 237. The second terminal of the pixel source follower transistor 237 is connected to a first terminal of the selection transistor 238. A signal (a pixel signal or a noise signal) converted into a voltage by the charge conversion unit 233 is input to the gate of the pixel source follower transistor 237. The pixel source follower transistor 237 outputs the imaging signal and the nose signal converted into the voltages by the charge conversion unit 233 to the vertical transfer line^{,} 239 via the selection transistor 238,

The selection transistor 238 includes a first terminal, a second terminal, and a gate. The first terminal and the second terminal of the selection transistor 238 are a source or a drain. The first terminal of the selection transistor 238 is connected to the second terminal of the pixel source fbllower transistor 237. The second terminal of the selection transistor 238 is connected to the vertical transfer line 239. A selection signal (not sliowi s supplied from the vertical scanning unit 241 to the gate of the selection transistor 238. The selection transistor 238 is switched on when receiving the selection signal from the vertical scanning unit 241. Thus, the selection transistor 238 is electrically connected to the pixel source follower transistor 237 and the vertical transfer line 239.

As described. above, two photoelectric conversion elements and two transfer transistors are included in one of the. pixels .230. One photoelectric conversion element z d one transti ..r transistor may be included in one of the pixels 230. Alternatively, three or more photoelectric conversion elements and three or more transfer transistors may he included in one of the pixels 230.

The current source 242 is constituted of a transistor. The current source 242 includes a first terminal, a second terminal, and a gate. The first terminal and the second terminal of the current source 242 are a source or a drain. The first terminal of the current source 242 is connected to the vertical transfer line 239. The second terminal of the current source 242 is connected to the ground. A.bias voltage Vbiasl is input to the gate of the current source 242, The current source 242 drives the pixels 230 and reads an imaging signal and a noise signal, which are output from the pixels 230, to the vertical transfer line 239. The imaging signal and the noise signal read to the vertical transfer line 239 are input to the noise removing unit 243,

The noise removing unit 243 includes a transfer capacitance 252 and a clamp switch 253. The transfer capacitance 252 includes a first terminal and a second terminal. The first terminal of the transfer capacitance 252 is connected to the vertical transfer line 239. The second terminal of the transfer capacitance 252 is connected to a gate of the column source follower buffer 244. The clamp switch 253 is a transistor. The clamp switch 253 includes a first terminal, a second terminal, and a gate. A clamp voltage Vclp is supplied from the reference voltage generation unit 246 to the first terminal of the clamp switch 253. The second terminal of the clamp switch 253 is connected to the second terminal of the transr capacitance 252 and the gate of the column source follower buffer 244. A control signal OVCI., is input .from the timing generation unit 25 to the gate of the clamp switch 253. from the timing generationmit 25 to the gate of the clamp switch 253. At this time, the transfer capacitance 252 is reset by the clamp voltage Yelp supplied from the reference voltage generation unit 246. The noise removing unit 243 generates an imaging signal corresponding to a difference between a pixel signal and a noise signal, In other words, the imaging signal from whiCh a noise component is removed is generated, The imaging signal from which the noise component is removed by the noise removing unit 243 is input to the gate of the column source follower buffer 241. With the above-described configuration, the noise removing unit 243 processes a pixel signal and outputs an imaging signal based on the pixel signal. The noise removing unit 243 functions as a pixel signal processing circuit.

The noise removing unit 243 does not require a sampling capacitor (a sampling capacitance). For this reason, the transfer capacitance has only to be sufficient for an input capacitance of the column source follower buffer 244. In addition, since there is no sampling capacitance, an area occupied by the noise oving unit 243 of the first chip 21 is small.

The column source follower buffer 244 is a transistor. The column source folio er buffer 244 includes a first terminal, a second terirainal, and a gate. The first terminal and the second terminal of the column source follower butler 244 are a source or a drain. The power supply voltage VDD is input to the first terminal of the column source follower buffer 244. The second terminal of the column source follower buffer 244 is connected to a first terminal of a col . selection switch .254. An imaging signal is input to the gate of the column source follower buffer 244 via the noise removing unit 243.

The column selection switch .254 is a transistor. The column selection switch 254 includes a first terminal, a second terminal, and a gate. The first terminal and the second terminal of the column selection switch. 254 are a source or a drain. The first terminal of the column selection switch 254 is connected to the second terminal of the column source follower buffer 244. The second terminal of the column selection switch 254 is connected to the horizontal transfer line 258. A control signal is supplied from the horizontal scanning unit 245 to the gate of the column selection switch 254. The column selection switch 254 is switched on when receiving the control signal 4HCLK<N>from the horizontal scanning unit 245. Thus, the column selection switch 254 outputs an imaging signal of the vertical transfer line 239 of a column <N>selected from the plurality of pixels 230 of the light receiving unit 23 to the horizontal transfer line 258 .

The level shift unit 249 is a resistor. The level shift unit 249 includes a first terminal and a second terminal. The first terminal of the level shift^{.} unit 249 is connected to the horizontal transfer line .258. The second terminal of the level shift unit 249 is connected to a second terminal of a horizontal reset transistor 256 and a first terminal of a constant current source 257. The level shift unit 249 shifts a first level of an imaging signal output to the horizontal transfer line 258 in a direction in which the first level is away from a second level of a reference signal Vref A voltage of the first terminal of the level shift unit 249 is higher than a voltage of the second terminal of the level shift unit 249. Therefore, the level shift unit 249 shifts the first level of the imaging signal output to the horizontal transfer line 258 in a lower level direction. The level shift unit 249 functions as a level shift circuit. The level shift unit 249 is disposed between the noise removing unit 243 and the buffer 26 in a transfer route of an imaging

The horizontal reset transistor 256 includes a first terminal, a second terminal, and a gate. The first terminal and the second terminal of the horizontal reset transistor 256 are a source or a drain. A horizontal reset voltage Ycir is input to the first terminal of the horizontal reset transistor 256. The second terminal of the horizontal reset transistor 256 is connected. to the second terminal of the level shift unit 249. A control signal (1)1-ICL.R. is input from the timing generation unit 25 to the gate of the horizontal reset transistor 256. The horizontal reset transistor 256 is switched on when receiving the control signal (1)HCLR from the timing generation unit 25. Thus, the horizontal reset transistor 256 resets the horizontal transfer line 258.

The constant current source 257 constitutes the constant current source 290. The constant current source 257 is a transistor. The constant current source 257 includes a first terminal, a second terminal, and a gate. The first terminal and the second terminal of the constant current source 257 are a source or a drain. The first terminal ofthe constant current source 257 is connected to the second terminal of the level shift unit 249. The second terminal of the constant current source 257 is connected to the ground. A bias voltage Vbias2 is input to the gate of the constant current source 257. The constant current source 257 drives the column source follower buffer 244 and reads an imaging signal from the vertical transfer line 239 to the horizontal transfer line 258. The imaging signal read to the horizontal transfer line 258 is input to the butler 26 via the level shill unit 249 and held. A detailed configuration of the reference voltage generation unit 246 will he described. The reference voltage generation unit 246 includes a resistor 291, a resistor 292, a switch 293, a sample capacitance 294, an operational amplifier 295, and an operational amplifier 296.

Each of the resistor 291 and the resistor 292 includes a first terminal and a second terminal. The power supply voltage VDD is input to the first terminal of the resistor 291. The second. terminal of the resistor 291 is connected to the first terminal of the resistor 292 and a first terminal of the switch 293. The first terminal of the resistor 292 is connected to the second terminal of the resistor 291 and the first terminal of the switch 293. The second terminal of the resistor 292 is connected to the ground. The resistor 291 and the resistor 292 constitute a resistance voltage-dividing circuit.

The switch 293 is a transistor. The switch 293 includes a first terminal, a second terminal, and a gate. The first terminal and. the second terminal f the switch 293 are a source or a drain. The first terminal of the switch 293 is connected to the second terminal of the resistor 291 and the first terminal of the resistor 292. The second terminal of the switch 293 is connected to a first terminal of the sample capacitance 294. A control signal OVSli is supplied from the timing generation unit 25 to the gate of the switch 293. The switch 293 is switched on when the control signal itiVSH is input from the. timing generationinit 25. Thus, the switch 293 outputs voltages according to resistance values of the resistor 291 and. the resistor 292 to the sample capacitance 294.

The sample capacitance 294 includes a first terminal and a second terminal.

The first terminal of the sample capacitance 294 is connected to the second terminal of the switch 293, a first terminal of the operational amplifier 295, and a first terminal of the operational amplifier 296. The second terminal of the sample capacitance 294 is connected to the ground. The sample capacitance 294 holds the voltages according to the resistance values of the resistor 291 and the resistor 292,

Each of the operational amplifier 295 and the operational amplifier 296 includes a first terminal and a second terminal. The first terminals of the operational amplifier 295 and the operational amplifier 296 are connected to the first terminal of the sample capacitance 294 and the second terminal of the switch 293. The second terminal of the operational amplifier 295 is connected to a gate of a pixel source follower transistor 237b.

The operational amplifier 295 outputs a voltage 1¹Td_H according to a voltage held in the sample capacitance 294 to the pixel source follower transistor 237b. The operational amplifier 296 outputs the clamp voltage Vclp according to the voltage held in the sample capacitance 294 from the second terminal thereof

With the above-described configuration, the reference voltage generation unit 246 generates the clamp voltage yelp and the voltage from the power supply voltage VDD at a timing according to the control signal 0¹S1-1. In other words, the reference voltage generation unit 246 generates the clamp voltage yelp (a reference voltage) used to operate the noise removing unit 243. Furthermore, the reference voltage generation unit 246 generates the voltage Vfd_I-1 used to operate the reference signal generation unit 248 . The reference voltage generation unit 246 functions as a reference voltage generation circuit. described. The reference signal generation unit 248 includes the pixel source follower transistor 237b. the current source 242a, the noise removing unit 243a, the column source follower buffer 244a, and the column selection switch 254a.

The pixel source follower transistor 237b includes the same configuration as the above-described pixel source follower transistor 237. The pixel source follower transistor 237b includes a first terminal, a second terminal, and a gate. The first terminal and the second terminal of the pixel source follower transistor 237b are a source or a drain. The power supply voltage VDD is input to the first terminal of the pixel source follower transistor 237b. The second terminal of the pixel source follower transistor 237h is connected to the vertical transfer line 239a. The voltage Vid El is input from the reference voltage generation unit 246 to the gate of the pixel source follower transistor 237b. The pixel source follower transistor 237b outputs a reference signal according to the voltage Vtd_{..}11 to the vertical transfer line 239a.

The current source 242a includes the same configuration as the above-described current source 242. The current source 242a is constituted of a transistor. The current source 242a includes a first terminal, a second terminal, and a gate. The first terminal and the second terminal of the current source 242a are a source or a drain. The first. terminal of the current source 242a is connected to the vertical transfer line 239a. The second terminal of the current source 242a is connected to the ground. The bias voltage Vbias1 is input to the gate of the current source 242a. The current source 242a drives the pixel source Ibllower transistor 2371 and reads the reference signal output from the pixel source follower transistor 237b to the vertical transfer line 239a. The reference signal read to the vertical transfer line 239a is input to the noise removing unit 243a. A noise eomponent is included in the refer nee signal input to the noise removing unit 243a.

The noise removing unit 243a includes the same configuration as the above-described noise removing unit 243. The noise removing unit 243a includes the transfer capacitance 252a and the clamp switch 253a, The transfer capacitance 252a includes a first terminal and a second terminal. The first terminal of the transfer capacitance 252a is connected to the vertical transfer line 239a, The second terminal of the transfer capacitance 252a is connected to a gate of the column source follower buffer 244a, The clamp switch 253a is a transistor. The clamp switch 253a includes a first terminal, a second terminal, and a gate. The clamp voltage Yelp is supplied from the reference voltage generation unit 246 to the first terminal of the clamp switch 253a. The second terminal of the clamp switch 253a is connected to the second terminal of the transfer capacitance 252a and the gate of the column source follower buffer 244a. The control signal (pV(.71, is input from the timing generation unit 25 to the gate the clamp switch 253a.

The clamp switch 253a is switched on when the control signal (WU is input from the timing generation tint 25 to the gate of the clamp switch 253a. At this time, the transfer capacitance 252a is reset by the clamp voltage Vclp supplied from the reference voltage generation unit 246 The noise removing unit 243a generates a reference signal from which a. noise component is removed. The reference signal from which the noise component is removed by the noise removing unit. 243a is input. to the gate of the column source follower buffer 244a. With the above-described configuration the noise removing unit 243a processes the reference signal and outputs an analog signal based on the reference signal. The noise removing unit 243a functions as a reference signal processing circuit.

The column source follower ItiMr 244a includes the same configuration as the above-described column source follower buffer 244, The column source follower buffer 244a is a transistor. The column source follower buffer 244a includes a first terminal, a second terminal, and a gate. The first terminal and the second terminal of the column source follower buffer 244a are a source or a drain. The power supply voltage VDD is input to the first terminal of the column source follower buffer 2443 The second terminal of the column source follower buffer 244a is onnected to a first terminal of the column selection switch 254a. The reference signal is input to the gate of the column source follower buffer 244a via the noise removing unit 243a. [0070]

The column selection switch 254a includes the same configuration as the above-described. column selection switch 2 The column selection switch 254a is a transistor. The column selection switch 254a includes a first terminal, a second terminal, and a gate . The first terminal and the second terminal of the column selection switch 254a are a source or a drain. The first terminal of the column selection switch 254a is connected to the second terminal of the column source follower buffer 244a, The second terminal of the column selection switch 254a is connected to the horizontal transfer line 258a. The control signal 0-1CLK<N>is supplied from the horizontal. scanning unit 245 to the gate of the column selection switch 254a. The column selection switch 254a is switched on When receiving the control signal OFICI ..K<N>^{.}from. the horizontal scanning unit 245. Thus, the column selection switch 254a outputs a reference signal of the vertical transfer line 239a to the horizontal transfer line 258a. The reference signal Vref output to the horizontal transfer line 258a is transferred to the butler 26.

The reference signal generation unit 248 has a structure which is equivalent to at least one among a plurality of circuits included in the imaging signal generation unit 240. To be specific, the reference signal generation unit 248 has a structure which is equivalent to that of the pixel source follower transistor 237, the current source 242, the noise removing unit 243, the column source follower buffer 244, and the column selection switch 254. in other words, the reference signal generation unit 248 includes the pixel source follower transistor 237b, the current source 242a, the noise removing unit 243a, the column source follower buffer 244a, and the column selection switch 254a which correspond to those of the above-described circuit.

With the above-described configuration, the reference signal generation unit 248 generates a reference signal Vref A common power supply voltage VDI) is supplied to the pixel source follower transistor 237, the column source follower buffer 244, the pixel source follower transistor 237b and the column source follower buffer 244a. A common bias voltage Vbiasi is supplied to the current source 24.2 and the current source 242a. A. common clamp voltage Vcip is supplied to the noise removing unit 243 and the noise removing unit 243a. For this reason, the reference signal Vref has a fluctuation component with the same phase as a fluctuation component of a power supply voltage which is present in an imaging signal generated by the imaging signal generation unit 240. [0073]

A level of the reference signal Vref is substantially the same as a level of the imaging signal generated by the imaging signal generation unit 240 when light of incident on the pixel 230. In other words, the level of the reference signal ‘Viet’ is substantially the same as a level of an imaging signal in the dark. Resistance values of the resistor 291 and the resistor 292 are set such that the level of the reference signal Vref is substantially the same as the level of the imaging signal in the dark. For this reason, the level of the reference signal Vref is substantially constant. A. voltage of the gate of the pixel source follower transistor 237b is close to a voltage of the gate of the pixel source follower transistor 237 in the dark. The voltage of the gate of the pixel source follower transistor 237b need not he the same as the voltage of the gate of the pixel source follower transistor 237 in the dark.

The constant current source 257a constitutes the constant current source 29f1. The constant current source 257a has the same configuration as the constant current source 257. The constant current source 257a is a transistor. The constant current source 257a includes a first terminal, a second tei minal, and a gate. The first terminal and the second terminal of the constant current source 257a are a source or a drain. The first terminal of the constant current source 257a is connected to the horizontal transfer line 258a. The second terminal of the constant current source 257a is connected to the ground. The bias voltage Vbias2 is input to the gate of the constant current source 257a, The constant current source 257a drives the column source follower buffer 244a and reads the reference signal Vref from the vertical transfer line 239a to the horizontal transfer line 258a, The reference signal Vref read to the horizontal transfer line 258a is input to the butler 26 and held.

The buffer 26 individually holds an imaging signal. input from the horizontal transfer line 258 and the reference signal Vref input from the horizontal transfer line 258a. The buffer 26 includes a signal output terminal 310 configured to output the reference signal Vref generated by the reference signal generation unit 248 and an imaging signal, a first level of which is shifted by the level shift unit 249, to the ME unit 51. The buffer 26 switches the reference signal \Tref and an imaging signal on the basis of a control signal 011iNSEL, from the timing generation unit 25. The reference signal Vref and the imaging signal output from the buffer 26 are output to the AFE unit 51 via the buffer 27 of the second chip 22. [0076] A detailed configuration of the buffer 26 will be described. The but x 26 includes a sample-and-hold unit 261, the multiplexer 263a, and an output buffer 3 I I. The sample-and-hold unit 261 includes a sample-and-hold switch 261e, a sample capacitance 261f an operational amplifier 261g, a resistor R1, and a resistor R2.

The sample-and-hold switch 261e is a transistor. The sample-and-hold switch 261 e includes a first terminal, a second terminal, and a gate. The first terminal and the second terminal of the sample-and-hold switch 261 e are a source or a drain. The first tenterminal ofthe sample-and-hold switch 261e is connected to the second terminal oaf the level shift unit 249. The second terminal of the sample-and-hold switch 261e is connected to a first terminal of the sample capacitance 261f and a non-inverting input terminal of the operational amplifier 261g. A control signal OffSli is supplied from the timing generation unit 25 to the gate of the sample-and-hold switch 261e. [0078]

The sample capacitance 261f includes the first terminal and a second terminal.

The first terminal of the sample capacitance 261f is connected to the second terminal of the sample-and-hold switch 26.1e and the non-inverting input terminal of the operational amplifier 261g. The second terminal of the sample capacitance 261.f is connected to the ground. The sample capacitance 261f holds a voltage of an imaging signal.

The operational amplifier 261g includes the non-inverting input terminal (+), an inverting input terminal (--), and an output terminal. The non-inverting input terminal of the, operational amplifier 261g is connected to the second terminal of the sample-and-hold switch 26Ie and the first terminal of the sample capacitance 261f The inverting input terminal of the operational amplifier 261g is connected to a first terminal of the resistor R1 and a second terminal of the resistor R2. The output terminal of the operational amplifier 261g is connected to the multiplexer 263a and a second terminal of the resistor RI. An imaging signal output from the output terminal of the operational amplifier 261; is input to the multiplexer 263a. .Furthermore, the imaging signal output from the output terminal of the operational amplifier 261a is input to the inverting input terminal of the operational amplifier 261₄₈ via the resistor R.1. in addition, the reference signal Vref from the reference signal generation unit 248 is input to the inverting input terminal of the operational amplifier 261 g via the resistor R.2. [0080]

The resistor R I includes the firstt terminal and the second terminal and the resistor R2 includes a first terminal and the second terminal. The first terminal of the resistor Rl is connected to the inverting input terminal of the operational amplifier 261.g and the second terminal of the resistor R2. The second terminal of the resistor RI is connected to the output. terminal of the operational. amplifier 261.g. The first. terminal of the resistor R2 is connected to the horizontal transfer line 258a. The second terminal of the resistor R2. is connected to the invertin input terminal of the operational amplifier 261g and the first terminal of the resistor R1 .

With the above-described configuration, the sample-and-hold unit 261 holds a voltage of an imaging signal in the sample capacitance 261f when the sample-and-hold switch 261e is switched on. The sample-and-hold unit 261 outputs the voltage held in the sample capacitance 261f to the operational amplifier 261g when the sample-and-hold switch 261e is switched off.

The multiplexer 263a outputs any one of the imaging signal output from the operational amplifier 261g and the reference signal Vref output from the reference signal generation unit 248 to the output buffer 31 on the basis of the control signal ONIUXS.EL input from the timing generation unit 2.5, The output buffer 31 includes a signal input terminal and the signal output terminal 3 10. The signal input terminal of the output buffer 31 is connected to the multiplexer 263a. The output buffer 31 alternately outputs the imaging signal and the reference signal Vref to the second Chip 21 With the above-described configuration, the buffer 26 functions as an output circuit configured to output an imaging signal and a reference signal. The second chip 22 transmits the imaging signal and the reference signal Vref to the connector unit 5 through the transmission cable 3.

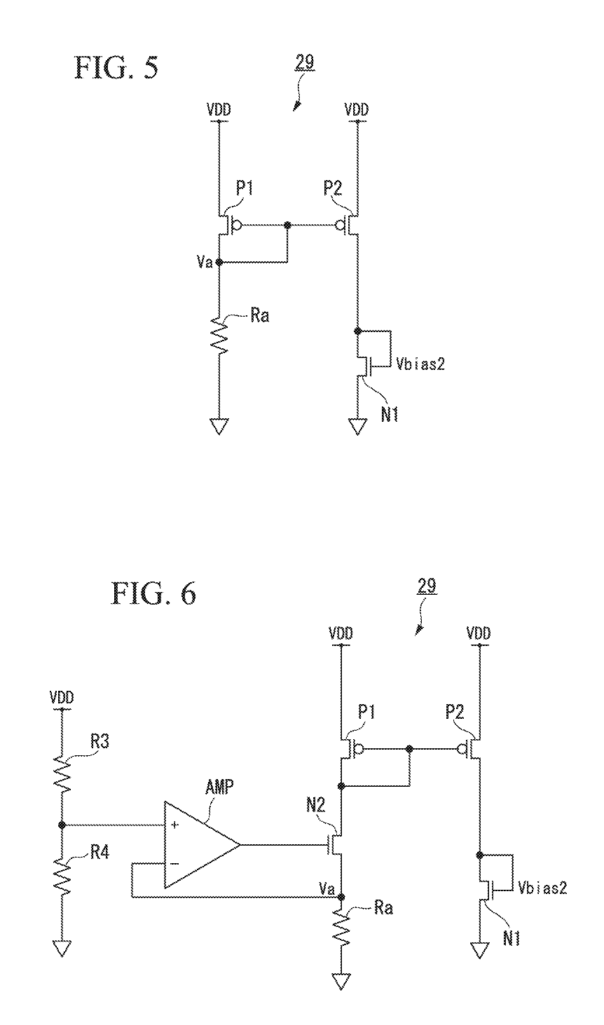

The reference current source 29 supplies an electric current to the constant current source 290. A detailed a configuration of the reference current source 29 will be described. Figs. 5 and 6 show the configuration of the reference current source 29. The configuration shown. in FIG. 5 is a first example of the configuration of the reference transistors P1. and P2, an N-type transistorNI, and a resistor Ra. The reference current source 29 constitutes a current mirror. The reference current source 29 outputs an electric current according to a voltage ‘Va of the resistor Ra The electric current value from the reference current source 29 is a value (Va/Ra) obtained by dividing the voltage Va by a resistance value (Ra) of the resistor .Ra.

The configuration shown in FIG. 6 is a second example of the configuration of the reference current source 29As shown in FIG. 6. the reference current source 29 includes P-type transistors PI and P2, N-type transistors Ni and N2, an operational amplifier AMP, and resistors Ra., R3, and. R4. The resistor R3 and the resistor R4 constitute a resistance voltage-dividing circuit. A voltage according to a ratio of resistance values of the resistor R3 and the resistor R4 is input to a non-inverting input terminal of the operational amplifier AMP. The operational amplifier AMP amplifies the voltage input to the non-inverting input terminal. The voltage output from the operational amplifier AMP is input to a gate of the transistor N2. The transistor N2 outputs an electric current according to the voltage input to the gate thereof to the resistor Ra. The reference current source 29 outputs an electric current according to the voltage Tia of the resistor Ra. The electric current value from the reference current source 29 is a value (Ya/Ra) obtained by dividing the voltage Va by the resistance value (Ra) of the resistor Ra.

The constant current source 257 gives an electric current, which is predetermined gain (a) times the electric current of the reference current source 29, to the column source follower buffer 244 via the level shift unit 249A. voltage Vr between the first terminal and the second terminal. of the level shift unit 249 is represented by Expression (1). In Expression (1). 8249 is a resistance value of the level shift unit 249.

Vr a x Va Ra x 8249 (1) [00861

The voltage Vr is a difference between a first level of. an imaging signal from the S pixels 230 and a level of an imaging signal, a first level of which is shifted by the level shift unit 249. The first level of the imaging signal in the dark is substantially the same as the level Of the reference signal Vref. Therefore, a difference between the level of the reference signal \lief and the level of the imaging signal, the first level of which in the dark is shifted by the level shift unit 249 is substantially the same as the voltage Vr. The voltage Vr is determined in accordance with a variation of the voltage Va and a mismatch between the resistor Ra and the level shift unit 249.

Since a voltage difference between the reference signal Vref and the imaging signal in the dark is small, such a voltage difference greatly influences accuracy of a signal processing. When the variation of the voltage Va is the same as a variation of the power supply voltage VDD, such a variation is 5% or less of that of the voltage Va. For example, a mismatch between resistance values of the resistor Ra and the level shift unit 249 is several percentages (for example, 3%). For this reason, a variation of the voltage V.r is 10% or less of that of the voltage Vr when there is no variation of the power supply voltage VDD and mismatch between the resistance values of the resistor Ra and the level.

shift unit 249. As a result, the variation of the voltage Vr is small. In other words, accuracy of a voltage difference between the reference signal Vref and the imaging signal in the dark is good. Theretbre calculation accuracy of a difference between the reference signal Vref and the imaging signal can be secured.

Transistor sizes of the column source f011ower but14 244 of the reading unit 24 and the col nn. source thilower buffer 244a of fhe reference signal generation unit 248 are substantially the same. Furthermore, bias electric current wlvalues of the column source follower buffer 244 of the reading unit 24 and the column source follower buffer 244a of the reference signal generation unit 248 are substantially the same. For this reason, a variation of a voltage difference between the imaging signal and the reference signal. Vref in accordance with the column source follower buffer 244 and the column. source follower buffer 244a can be minimized. In other ords, accuracy of the voltage difference between the reference signal Vref and the imaging signal in the dark is better.

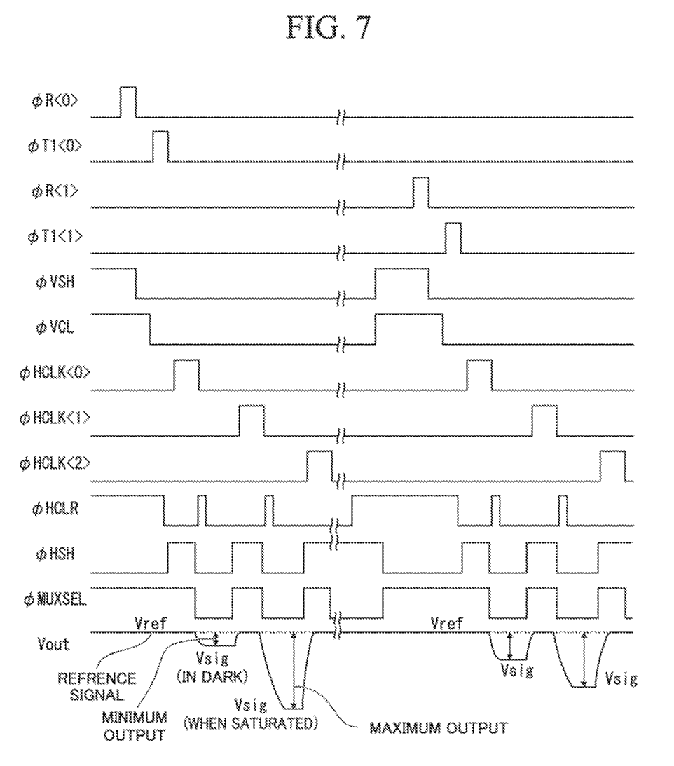

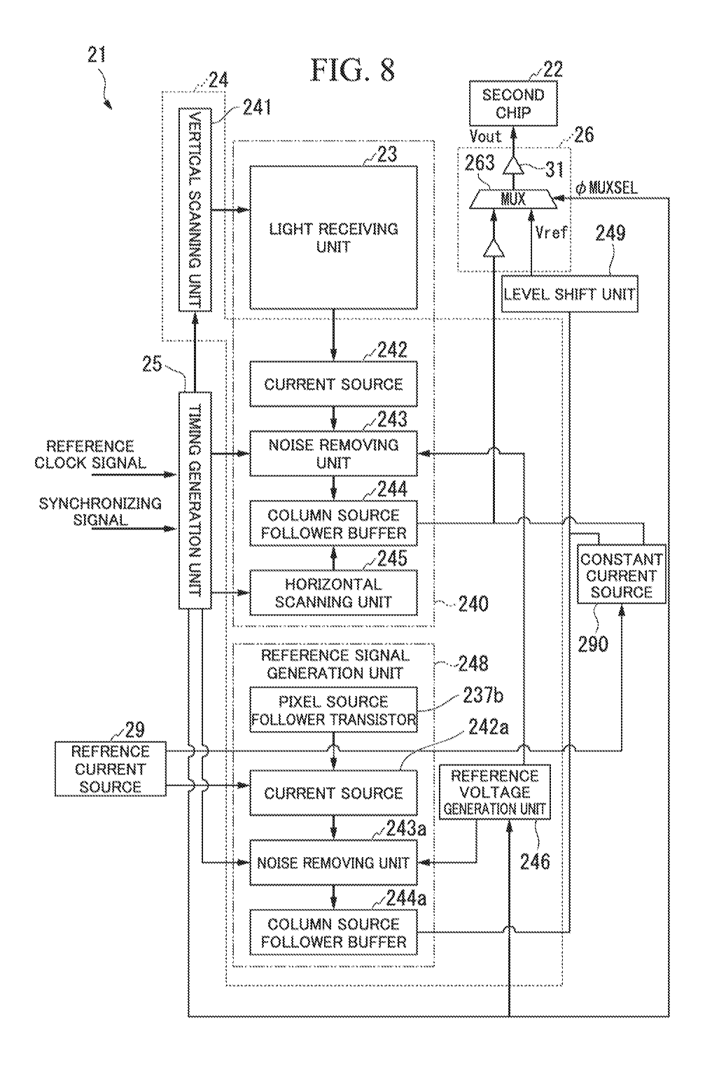

A drive timing ofthe imaging unit 20 will be described. FIG. 7 shows an operation of the imaging unit 20. FIG. 7 shows waveforms of a control signal R<0>. a control signal (lal <0>a control signal 4)R <l>a control signal clal<l>, a control signal

(INSFI, a control signal 00, a control signal HC.E.K<O>, a control signal (1)110,,K.<1>, a control signal 01(1,,K<2.-- a control signal (141(.71,,R, a control signal F1Slt, a control signal iliMUX.SEL, and an output voltage Vout. The output voltage Vout is a voltage of the signal output terminal 310 of the output buffer 31. In FIG. 7, the horizontal direction indicates time and the vertical direction indicates a voltage. [00901 An operation of reading a signal from a row<>and a row<1>of the plurality of the. pixels 230 and an operation of outputting the read signal from the output buffer 31 will be described e with reference to Fig. i FIG. 7 shows an operation when the^{,} photoelectric conversion element 231 is included in the pixel 230 and the photoelectric conversion element 232 is not included in the pixel 230 for convenience of explanation. When a plurality of photoelectric conversion elements are included in. the pixel 230, an.

operation tbr one line shown in FIG. 7 is repeated by the number of photoelectric conversion elements included in the pixel 230. With regard to the control signal 4li and the control signal iVfl of FIG. 7, signals corresponding to the ^{.}row<O>and the ow<l>are^{,} shown. :Furthermore, with regard to the control signal (liFICIK of FIG. 7, signals corresponding to a column’ l>and a column<2>are shown. [0091]

As shown in Fig, 7 the control signal ktiVCI., becomes a High level so that the clamp switch 253 is switched on. A pulsed control signal 4)R<O>becomes a .High level so that the pixel reset transistor 236 is switehed on. Thus, a noise signal including a variation peculiar to the pixel 230 and a noise when the pixel 230 is reset is output from the pixel 230 to the vertical transfer line 239. The clamp switch 253 is kept switched on so that a gate voltage of the column source follower buffer 244 becomes the clamp voltage Yelp. The clamp voltage Velp is fixed at a tinting at which the control signal 4NS14 changes from a High level to a Low level. [009.2]

The clamp switch 253a is switched on at a timing at which the clamp switch 253 is switched on. The voltage Vtd J1 from the reference voltage generation unit 246 is fixed at a timing at which the control signal 4VSH changes from the High level to Low level,. [0093]

The control signal 4NCI., becomes a Low level so that the clamp switch 253 is switched off. A pulsed control signal 01<0>becomes a High level so that the transfer transistor 234 is switched on in a pluse tbrm. Thus, an imaging signal based on a voltage of the chat - conversion unit 233 is read from the pixel 230 to the vertical. transfer line 239. The voltage of the charge conversion unit 233 is based onan electric charge transferred from each of the photoelectric conversion elements 231. With such an operation, an imaging signal is output to the gate of the column source follower butler 244 via the transfer capacitance 252. [00941

The imaging signal output to the gate of the column source follower buffer 244 is a signal which is sampled using the clamp voltage Yelp as a reference. hi other words, the imaging signal output to the gate of the column source follower buffer 244 is a signal from which a noise component is removed. [00951 The imaging signal is sampled using the clamp voltage Yelp as the reference and then the control signal 4)HCLIZ becomes a Low level so that the horizontal reset transistor 256 is switched off. Thus, the resetting of the horizontal transfer line 258 is released. [00961

Subsequently, a pulsed control signal ilitICLK<O>becomes a High level so that the column selection switch 254 of a column 0′>is switched on. Thus, an imaging signal of the column O>is transferred to the horizontal transfer line 258.

Simultaneously, a pulsed control signal FISH becomes a High level so that the sample-and-hold Switch 261 e is switched on ill a pulse form. Thus, the imaging signal is sampled. in the sample capacitance 261f via the level shift unit 249 and the sample-and-hold switch 261.e.

Subsequently, the control signal (1)111:UXSEI., with a Low level is input to the multiplexer 263a. Thus, the imaging signal sampled in the sample capacitance 261f is output to the output buffer 31. A pulsed control signal 44.1CLR becomes a:High level at a timing at which the control signal ONIUXSEL becomes a Low level so that the horizontal reset transistor 256 is switched on. Thus, the horizontal transfer line 258 is reset. again. In addition, the control signal 0I-ICLR. becomes a Low level so that the horizontal reset transistor 256 is switched off. Thus, the resetting of the horizontal transfer line 258 is released. [00981

Subsequently, the control signal OvIIASEL with a :High level is input to the multiplexer 263a, Thus, the reference signal Vref generated by the reference signal generation unit 248 is output to the output butler 31. [0099] Subsequently, the control signal 4)FICLK<I>becomes a High level so that the column selection switch 254 of the column<r,- is switched on, Thus, an imaging signal of the column<l>is transfened to the horizontal transfer line 258, Simultaneously the pulsed control signal (141SH becomes a High level so that the sample-and-hold switch 261e is switched on in a pulse form. Thus, the imaging signal is sampled in the sample capacitance 261 f via the level shift unit 249 and the sample-and-hold switch 261e . [0100]

Subsequently, the control signal 4A11,1XSEL with a Low level is input to the multiplexer 263a. Thus, the imaging signal which is sampled. in the sample capacitance 261f is amplified by the operational amplifier 261g and output to the output buffer 3 I The pulsed control signal 4)H. CLR becomes a High level at a timing at which the control signal OMUKSEL becomes a Low level so that the horizontal reset transistor 256 is switched on. Thus, the horizontal transfer line 258 is reset again. In addition, the control signal IITR becomes a tow level so that. the horizontal reset transistor 256 is switched off. Thus the resettit of the horizontal transfer line 258 is released.

[01011

After imaging signals A. all of the pixels 230 of the row<0>are transferred to the horizontal transfer line 258, the control signal (INSEI and the control signal Oa, become Ifigh levels. Thus, the transferring of the imaging signals of the row<0>is completed, and transferring of imaging signals of the row<l>is started. [01021

The above-described operation is repeated by the number of columns (or the number of columns to be read) of the plurality of the pixels 230. ‘Thus, the imaging signal and the reference signal Vref are alternately output from the output butler 31. reading operation for one line is repeated by the number of rows (car the number of rows to be read) of the plurality of the pixels 230 so that imaging signals and reference signals ‘rel for one frame are output [01031

A control signal supplied to the selection transistor ⁷38 is not shown ire FIG. 7. When a noise signal or a pixel signal is read from the pixel 230, the selection transistor 238 is switched on. [01041 hi FIG. 7, an imaging signal Vsig of the row<O>and the column<O>is a signal generated when light is not incident on the pixels 230 (in the dark). At this time, a difference between the reference signal Vref and the imaging signal V.7 g is a minimum output. In Fig,, 7. an imaging signal Vsig of the row<O>and the col Mill<>is a signal generated when light by which the photoelectric conversion element 231 is saturated is incident on the pixels 230 (when saturated). At this time, a difference between the reference signal Vref and the imaging signal Vsig is the maximum output. [01051

A relationship between magnitudes of levels of the reference signal Vref and the imaging signal is constant regardless of whether light is incident on the pixels .230 . For example, in FIG. 7, a level of an imaging signal when light is not incident on. the pixels 230 is smaller than the level of the reference signal Vref, Similarly, a. level of an imaging signal when light is incident on the pixels 230 is smaller than the level of the reference signal Vref In other words, a level of an imaging signal is smaller than the level of the reference signal. Vref at all times, As described above, a relationship between magnitudes of levels of the reference signal Vref and the imaging signal output from the signal output terminal 310 when the light is not incident on the plurality of the pixels 230 is the same as a relationship between magnitudes of levels of the reference signal Vref and the imaging signal output from the signal output terminal 310 when the light is incident on the plurality of the pixels 230, .For this reason, the .AFE unit 51 can correctly process the reference signal Vref and the imagine signal. [01061

A difference between the levels of the reference signal Vref and the imaging signal is larger than 0. When the light is not incident on the plurality of the pixels 230, the difference between the levels of the reference signal Vref and the imaging signal is a minimum. As the light which is incident on the plurality of the pixels 230 increases, the difference between the levels of the reference signal Vref and the imaging signal increases. As the difference between the levels of the reference signal Vref and the imaging signal increases, accuracy of the difference between the levels of the reference signal Vref and the imaging signal is improved. On the other hand, as the difference between the levels of the reference signal Vref and the imaging signal when the light is not incident on the plurality of the pixels 230 decreases, a dynamic range in the An: unit. 51 increases, [01071 A design value of an amount of shift of an imaging signal level is determined by considering accuracy of a ditkrence and a dynamic range. For example_{:}, a difference between levels of the reference signal \Tref and the imaging signal output from the signal output terminal 310 when light is not incident on the plurality of the pixels 230 is within 20% of the maximum value of a difference between levels of a reference signal Vref and an imaging signal which can be output from the signal output terminal 310. The diftWence between the levels of the reference signal Vref and the imaging signal output from the signal output terminal 310 when the light is not incident on the plurality of the pixels 230 is a minimum output in Fig, 7. ‘The maximum value of the difference between the levels of the reference signal Vref and the imaging signal which can be ouput from the signal output terminal 310 is the maximum output in FIG. 7. [0108]

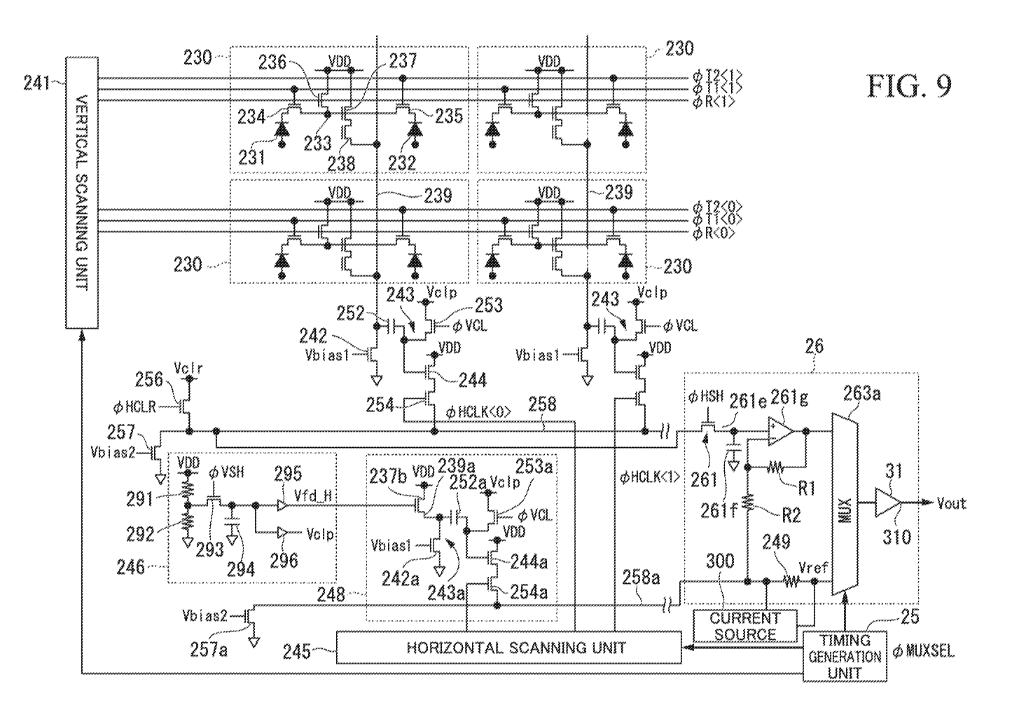

(Modified example) FIG. 8 shows a configuration of a first chip 21 in a modified example according to the embodiment of the present invention. FIG. 9 shows a circuit configuration of the first chip 21 in the modified example according to the embodiment of the present invention, As shown in Figs, 8 and 9 . the first chip 21 includes a light receiving unit 23, a reading unit 24, a timing generation unit 25, a buffer 26, a reference current source 29. and a constant current source 290. [0109]

In FIGS. 8 and 9, a configuration other than a configuration associated with the level shift unit 249 is the same as the configuration shown in FIGS. 3 and 4. Only the configuration associated with the level shift unit 249 will be described below, and a description of other configurations will. be omitted. [0110]

A first terminal. of a level. shift unit 249 is connected to a horizontal transfer line 258a. .A second terminal of the level shift unit 249 is connected tto a multiplexer 263a. The level shift unit .249 shifts a second level of a reference signal Vref in a direction in. which the second level is away from a first level of an imaging signal. A voltage of the second terminal of the level shift unit 249 is higher than a voltage of the first terminal of the level shift unit 249. Therefore, the level shift unit 249 shifts a second level of the reference signal vief output to the horizontal transfer line 258a in a higher level direction, The level shift unit 249 functions as a level shift circuit, The level shift unit 249 is disposed between a reference signal generation unit 248 and the buffer 26 in a transfer route of the reference signal Well [0 H 11

A current source 300 supplies an electric current to the first terminal and the second terminal of the level shift unit 249, A second terminal of a horizontal reset transistor 256, a first terminal of a constant current source 257, and a first terminal of a sample-and-hold. switch 261 e are connected to a horizontal traiisr line 25$. [01121

As described above, the imaging unit 20 (the imaging device) according to the einbodim of the present invention includes the plurality of the pixels 230, the noise removing unit 243 (the pixel signal processing circuit), the reference signal generation unit 248 (the reference signal generation circuit), the level shift unit 249 (the level shift circuit), and the signal output terminal 310. The plurality of the pixels .230 outputs pixel signals. A noise removing unit 243 processes a pixel signal and outputs an imaging signal based on the pixel signal. The imaging signa 1 based on the pixel signal is an imaging signal from which a noise component is removed. The reference sign a1 generation unit 248 generates the reference signal. Vref. The level shift unit 249 shifts the first level of the imaging signal in a direction in which. the first level thereof is away from the second level of the reference signal Vref Alternatively, the level shift unit 249 shills the second level in a direction in which the second level is away from the first level. A signal Output terminal 310 outputs the reference signal Vref generated by the reference signal generation unit 248 and the imaging signal, a first level of which is shifted by the level shill unit 249, to an ME unit 51. (an imaging signal processing circuit). Alternatively, the signal output terminal 310 outputs the reference signal Vref, a second level of which is shifted by the level shift unit 249, and the imaging signal output from the noise removing unit 243. The AFE unit 51 calculates a difference between the reference signal Vref and the imaging signal output from the signal output terminal 310.

The imaging devices of aspects of the present invention may not include at least one of a configuration other than a plurality of the pixels 230, a noise removing unit 243, the reference signal generation unit 248, the level shift unit 249, and the signal output terminal 310.

[01141

The endoscope 2 according to the embodiment of the present invention includes the insertion unit 100 inserted into a subject. The imaging unit 20 is disposed, on a tip of the insertion unit. 100, [0115]

The endoscope system 1 according to the embodiment of the present invention includes the endoscope 2, the NEE unit 51. (the imaging signal processing circuit), and the image signal processing unit 62 (the image signal generation circuit). The image signal processing unit 62 processes a diference signal based on the difference calculated by the AFE unit 51 and generates the image signal based on the diference signal. [011.61

The endoscope system of aspects of the present invention may not include at least one of a configuration other than the endoscope 2, the AFF, unit 51, and the image signal processing unit 62. [01171

In the embodiment of the present invention, calculation accuracy of the difference between the reference signal Wet. and the imaging signal can be secured using a function of the level shift unit 249. [01181 As described above, the variation of the voltage difference between the imaging signal and the reference signal Vref in accordance with the column source follower buffer 244 and the column source follower buffer 244a can be minimized. in other words, accuracy of the voltage difference between the reference signal Vref and the imaging signal in the dark. is better. [01191

The difference between the levels of the reference signal Vref and the imaging signal when the light is not incident on the plurality of the pixels 230 is within 20% of the maximum value of the difference between the levels of the reference signal Vrefand the imaging signal which can be output from the signal output terminal 310, For this reason, a. dynamic range of the AFT. unit 51 is secured and a decrease in. signal-to-noise

(SIN) of a signal output from the AFT unit 51 is suppressed . When an amount of noise is constant. an S/N of a signal depends on a m.anufacturing variation of a. transistor or the like. An SIN of a signal in such a case is improved by 2 to 3 d13 compared to that of a case in which the difference between the levels of the reference signal Wet’ and the imaging signal. when the I ght is not incident on the plurality of the pixels 230 is within.

40% of the maximum value of the difference.

[01.2011

When a fluctuation component (a ripple component) having the same phase as a fluctuation component of a power supply voltage is superimposed on a level of an. image signal, an .AFE circuit in the related art cannot remove the fluctuation component superimposed on the image signal. For this reason, an image quality deteriorates. On the other hand, the reference signal ‘ref according to the embodiment of the present invention is generated from the reference voltage used to operate the noise removing unit 243. For this reason, the reference signal Vref includes a fluctuation component having. the same phase as a fluctuation component of a power supply voltage which is present in an imaging signal. As a result, a fluctuation component in a diference signal based on a difference between a reference signal and the imaging signal which are calculated by the AFE unit 51 is reduced. Therefore, deterioration of an image quality is suppressed. [01211 While preferred embodiments of the invention have been described and shown above, it should be understood that these are exemplary of the invention and are not to be considered as limiting . Additions, omissions, substitutions, and other modifications can be made without departing from the spirit or scope ofthe present invention. Accordingly, the invention is not to he considered as being limited by the .foregoing description, and is only limited by the scope of the appended claims.

Viliat is claimed is:

1. An imaging device, comprising:
a plurality of pixels configured to output pixel signals;
a pixel signal processing circuit configured to process each of the pixel signals and output an imaging signal based on the pixel signal;
a reference signal generation circuit configured to generate a reference signal;
a level shift circuit configured to shift a first level of the imaging signal in a direction in which the first level is away from a second level of the reference signal or shift the second level in a direction in which the second level is away from the first level;
and a signal output terminal configured to output the reference signal generated by the reference signal generation circuit and the imaging signal, the first level of which is shifted by the level shift circuit, to an imaging signal processing circuit, or output the reference signal, the second level of which is shifted by the level shift circuit, and the imaging signal output from the pixel signal processing ircuit to the imaging signal processing circuit, wherein the imaging signal processing circuit calculates a difference between the reference signal and the imaging signal output from the signal output terminal .

2. The imaging device according to claim 1, wherein a relationship between levels of the. reference signal and the imaging signal output from the signal output terminal when light is not incident on the plurality of pixels is the same as a relationship between levels of the reference signal and the imaging signal output from the signal output terminal when. light is incident on the plurality of pixels,

3. The imaging device according to claim 1, wherein a difference of levels of the reference signal and the imaging signal output from the signal output terminal when light is not incident on the plurality of pixels is within 20% of the maximum value of a difference between levels of the reference signal and the imaging signal which can he output from the signal output terminal.

4. The imaging device according to claim 1, further comprising:
a reference voltage generation circuit configured to generate a reference voltage used to operate the pixel signal processing circuit, wherein the reference signal generation circuit generates the reference signal from the reference voltage.

5. An endoscope, comprising:
an insertion unit configured to be inserted into a subject; and
the imaging device according, to Claim I disposed on a tip of the insertion unit.

6. An endoscope system, comprising:
the endoscope according to claim 5;
the imaging signal processing circuit; and
an image signal generation circuit configured to process a difference signal based on the. differe. ce. calculated by the imaging signal processing circuit and generate an image signal based on the difference signal.
